(19) United States
(12) Patent Application Publication
(10) Pub. No.: US 2026/0015658 A1
(43) Pub. Date: Jan. 15, 2026

(54) Method of Nucleic Acid Sequencing

(71) Applicants: SEQULITE GENOMICS US, INC., Dublin, CA (US); Guangdong Runpeng Biological Technology Co., LTD., Dongguan City (CN)

(72) Inventor: Xiongwei YAN, Dublin, CA (US)

(21) Appl. No.: 18/770,573

(22) Filed: Jul. 11, 2024

(51) Int. Cl. C12Q 1/6874; C12Q 1/25; C12Q 1/34; C12Q 1/6825; C12Q 1/6834; C12Q 1/6844

(52) U.S. Cl. CPC C12Q 1/6874; C12Q 1/25; C12Q 1/34; C12Q 1/6825; C12Q 1/6834; C12Q 1/6844; G01N 2333/9015; G01N 2333/924

(57) ABSTRACT

Method of nucleic acid sequencing is provided, including: S1, providing a solid support with at least two types of nucleic acid linkers and a single-stranded template nucleotide, where both ends of the single-stranded template nucleotide have sequences that are complementary to the nucleic acid linkers and the single-stranded template nucleotide has a region to be detected, respectively, and the region to be detected includes a first region to be detected and a second region to be detected; S2, forming multiple nucleotide strands identical or complementary to the single-stranded template nucleotide on the solid support by a bridge amplification reaction, removing one of the nucleotide strands identical or complementary to the single-stranded template nucleotide; and S3, hybridizing sequencing primers of the first region to be detected and the second region to be detected on the first sequencing strand, and sequencing in segments or extending and linking into a whole synthetic strand.

### TECHNICAL FIELD

The present disclosure relates to the technical field of nucleic acid sequencing, in particular to a method of nucleic acid sequencing.

### BACKGROUND

Second-generation sequencing technology, also known as next generation sequencing technology (NGS), is also called high-throughput sequencing technology due to its high throughput, which can read thousands of short DNA fragments at one time.

Classical second-generation sequencing mainly includes three steps: library preparation, bridge amplification, and sequencing. The library preparation includes: 1) randomly breaking the DNA to be detected into fragments of a certain length; 2) the both ends of the fragments are processed. As shown in FIG. 1, each resulting DNA single strand has P7, i7 index at the 5′ end, and Rd2SP (Read2 Sequencing Primer), and P5′, i5 index, and Rd1SP (Read1 Sequencing Primer) at the 3′ end. These DNA single strands form a sample library. Numerous oligonucleotide strands (P5, P7′) are immobilized on the surface of the microarray chip used for sequencing, which can complement and bind to P5′ and P7, respectively, so that the sample library is immobilized on a sequencing platform, and subsequent sequencing by synthesis is performed.

At present, different sequencing platforms have different sequencing strategies. In order to provide more and more flexible sequencing possibilities, technicians are still developing sequencing strategies.

### SUMMARY

The present disclosure aims to provide a method of nucleic acid sequencing to provide more and more flexible sequencing possibilities.

In order to achieve the above purpose, according to one aspect of the present disclosure, a method of nucleic acid sequencing is provided. The method includes: S1, providing a solid support with at least two types of nucleic acid linkers and a single-stranded template nucleotide, where both ends of the single-stranded template nucleotide have sequences that are complementary to the nucleic acid linkers and the single-stranded template nucleotide has a region to be detected, respectively, and the region to be detected includes a first region to be detected and a second region to be detected; S2, forming multiple nucleotide strands identical or complementary to the single-stranded template nucleotide on the solid support by a bridge amplification reaction, removing one of the nucleotide strands identical or complementary to the single-stranded template nucleotide by cleaving, and retaining a first sequencing strand; and S3, hybridizing sequencing primers of the first region to be detected and the second region to be detected on the first sequencing strand, and sequencing in segments or extending and linking into a whole synthetic strand.

Furthermore, the synthetic strand is immobilized to the solid support by a 5′ end or a 3′ end.

Furthermore, when the synthetic strand is immobilized on the solid support by the 5′ end, the S3 includes: hybridizing a 3′ end of the first sequencing strand to the nucleic acid linkers on the solid support; hybridizing the sequencing primers of the first region to be detected and the second region to be detected on the first sequencing strand, and sequencing in segments or extending and linking into the whole synthetic strand, where the linking includes linking the nucleic acid linkers on the solid support to other segments of the synthetic strand.

Furthermore, when the synthetic strand is immobilized on the solid support by the 3′ end, the S3 includes: hybridizing the sequencing primers of the first region to be detected and the second region to be detected and a free 3′ end amplification primer on the first sequencing strand, and sequencing in segments or extending and linking into the whole synthetic strand; and immobilizing the 3′ end of the synthetic strand on the solid support by a cross-linking reaction.

Furthermore, the method of nucleic acid sequencing is a double-ended sequencing method, the first region to be detected is a sequence to be detected with an unknown sequence, and the second region to be detected is a first index region or a second index region.

Furthermore, in the S3, the step of hybridizing sequencing primers of the first region to be detected and the second region to be detected on the first sequencing strand, and sequencing in segments or extending and linking into a whole synthetic strand includes: phosphorylating a 5′ end of the sequencing primer of the first region to be detected, and phosphorylating both a 3′ end and a 5′ end of the sequencing primer of the second region to be detected; performing partial sequencing on the first region to be detected using a reversible terminator, preferably with a length of 35-150 bp for the partial sequencing; extending the first sequencing strand as a template to form a segmented synthetic strand using a nucleic acid linker complementary to the 3′ end of the first sequencing strand on the solid support or the free 3′ end amplification primer; linking the segments of the synthetic strand; and dephosphorylating the 3′ end of the sequencing primer of the second region to be detected, and sequencing or extending the second region to be detected to form the synthetic strand.

Furthermore, the single-stranded template nucleotide sequentially includes a first linker sequence region linked to the solid support, a first index region, a first sequencing primer region, a sequence region to be detected, a second sequencing primer region, a second index region, and a second linker sequence region linked to the solid support from a 5′ end to a 3′ end; and the method includes: immobilizing the single-stranded template nucleotide on the solid support through a bridge reaction to form a cluster, removing one of nucleotide strands identical or complementary to the single-stranded template nucleotide by cleaving, and retaining a first sequencing strand; hybridizing a probe carrying a fluorescent label for identifying the first index region with a nucleic acid of the first index region to collect a fluorescent signal and identify a sequence of the first index region, and then stripping the probe; hybridizing a first nucleic acid fragment covering a portion of the first linker sequence region, the first index region and the first sequencing primer region with the cluster, linking the first nucleic acid fragment to a linker sequence on the solid support, and partially sequencing the sequence to be detected with the first nucleic acid fragment as a primer, and then extending to the second linker sequence region; removing the first sequencing strand; hybridizing a probe carrying a fluorescent label for identifying the second index region with a nucleic acid of the second index region to collect a fluorescent signal and identify a sequence of the second index region, and then stripping the probe; and performing complementary strand sequencing of the sequence to be detected using a second nucleic acid fragment complementary to the second sequencing primer region as a primer.

Furthermore, the removing one of nucleotide strands identical or complementary to the single-stranded template nucleotide by cleaving includes: cleaving a uracil base or an 8-oxoguanine base on a nucleic acid linker on the solid support to form a nick, and removing a nucleotide strand with the nick using a formamide solution.

Furthermore, the uracil base is cleaved using a uracil hydrolase, or the 8-oxoguanine base is cleaved using an Fpg glycosidase.

Furthermore, both a 5′ end and a 3′ end of the first nucleic acid fragment are phosphorylated.

Furthermore, the step of linking the first nucleic acid fragment to a linker sequence on the solid support, and sequencing the sequence to be detected with the first nucleic acid fragment as a primer specifically includes: linking the first nucleic acid fragment to the linker sequence on the solid support using a DNA ligase, removing phosphorylation of the 3′ end of the first nucleic acid fragment using a phosphatase, and sequencing the sequence to be detected with the first nucleic acid fragment as a primer.

Furthermore, after the removing the first sequencing strand, the method further includes a step of blocking a free 3′-hydroxyl group with a terminator or phosphorylation, and optionally, the terminator is a dideoxynucleotide.

Furthermore, sequences of the probe for identifying the first index region and the probe for identifying the second index region are known, and the probe each carries a preset fluorescent label.

Furthermore, the step of hybridizing a probe carrying a fluorescent label for identifying the first index region with a nucleic acid of the first index region to collect a fluorescent signal and identify a sequence of the first index region, or the step of hybridizing a probe carrying a fluorescent label for identifying the second index region with a nucleic acid of the second index region to collect a fluorescent signal and identify a sequence of the second index region includes: S1, dividing the probe into N group(s), with the N≥1, where each group of the probe includes a type(s) of the probe, with the a≥1, and the a type(s) of the probe in the group each carries a different fluorescent label; and hybridizing a first group of the probe carrying a fluorescent label with the nucleic acid of the first index region to collect a fluorescent signal; S2, stripping the first group of the probe, and hybridizing a second group of the probe with the nucleic acids of the first index region or the second index region to collect a fluorescence signal; and S3, repeating the step S2 until all the first index region or the second index region is detected.

Furthermore, stripping the probe includes stripping the probe with a formamide solution; and preferably, it further includes determining whether the probe is completely stripped by fluorescence observation.

According to another aspect of the present disclosure, a nucleic acid sequencing kit is provided. The nucleic acid sequencing kit includes a relevant reagent for nucleic acid sequencing and a DNA ligase; and preferably, the relevant reagent for nucleic acid sequencing includes a sequencing primer, a polymerase, and dNTP.

According to another aspect of the present disclosure, a nucleic acid sequencing apparatus is provided. The nucleic acid sequencing apparatus includes a control unit and a sequencing unit, where the control unit controls the sequencing unit to perform any one of the above methods of nucleic acid sequencing.

Through applying the technical solutions of the present disclosure, the sequencing synthetic strand is synthesized in segments and linked to form a whole synthetic strand, which is a more flexible sequencing method and can be used in combination with other non-sequencing index detection methods. In addition, in conventional sequencing, after measuring the target sequence and index, respectively, the synthetic strand needs to be stripped and removed, and then several steps are taken to complete the sequence turnaround commonly used in paired end sequencing, which are relatively cumbersome; and in this disclosure, there is no need for synthetic strand removal and sequence turnaround, and the steps are simple.

### BRIEF DESCRIPTION OF THE DRAWINGS

The accompanying drawings of the specification, which form part of this disclosure, are used to provide a further understanding of the present disclosure, and the illustrative embodiments and their explanations of the present disclosure are used to explain the present disclosure and do not constitute an improper limitation of the present disclosure. In the drawings:

FIG. 1 shows a schematic diagram of a single-stranded template nucleotide structure according to an embodiment of this disclosure;

FIGS. 2-1 and 2-2 show a schematic diagram of a sequencing process according to an embodiment of this disclosure;

FIGS. 3-1, 3-2, and 3-3 show a schematic diagram of a sequencing process according to another embodiment of this disclosure;

FIGS. 4-1, 4-2 and 4-3 show a schematic diagram of a sequencing process according to yet another embodiment of this disclosure; and

FIG. 5 shows relationships between a cluster average fluorescence intensity and an SBS cycle for four channels.

### DETAILED DESCRIPTION OF THE EMBODIMENTS

It should be noted that, in the absence of a conflict, embodiments of this disclosure can be combined with features in the embodiments. The present disclosure will be described in detail below with reference to the accompanying drawings and in conjunction with embodiments.

As described in the background art of this disclosure, different sequencing platforms have different sequencing strategies. In order to provide more and more flexible sequencing possibilities, technicians are still developing sequencing strategies. In this disclosure, the inventor proposes a more flexible sequencing method, and the specific technical solutions are described as follows:

According to a typical implementation of this disclosure, a method of nucleic acid sequencing is provided. The method includes: S1, providing a solid support with at least two types of nucleic acid linkers and a single-stranded template nucleotide, where both ends of the single-stranded template nucleotide have sequences that are complementary to the nucleic acid linkers and the single-stranded template nucleotide has a region to be detected, respectively, and the region to be detected includes a first region to be detected and a second region to be detected; S2, forming multiple nucleotide strands identical or complementary to the single-stranded template nucleotide on the solid support by a bridge amplification reaction, removing one of the nucleotide strands identical or complementary to the single-stranded template nucleotide by cleaving, and retaining a first sequencing strand; and S3, hybridizing sequencing primers of the first region to be detected and the second region to be detected on the first sequencing strand, and sequencing in segments or extending and linking into a whole synthetic strand.

Through applying the technical solutions of the present disclosure, the sequencing synthetic strand is synthesized in segments and linked to form a whole synthetic strand, which is a more flexible sequencing method and can be used in combination with other non-sequencing index detection methods. In addition, in conventional sequencing, after measuring the target sequence and index, respectively, the synthetic strand needs to be stripped and removed, and then several steps are taken to complete the sequence turnaround commonly used in paired end sequencing, which are relatively cumbersome; and in this disclosure, there is no need for synthetic strand removal and sequence turnaround, and the steps are simple.

In the disclosure, the synthetic strand can be immobilized to the solid support in different ways, such as by a 5′ end or a 3′ end.

Preferably, when the synthetic strand is immobilized on the solid support by the 5′ end, the S3 includes: hybridizing a 3′ end of the first sequencing strand to the nucleic acid linkers on the solid support; hybridizing the sequencing primers of the first region to be detected and the second region to be detected on the first sequencing strand, and sequencing in segments or extending and linking into the whole synthetic strand, where the linking includes linking the nucleic acid linkers on the solid support to other segments of the synthetic strand. Since the nucleic acid linkers on the solid support are originally immobilized on the solid support, only the subsequent synthesized part needs to be linked to the nucleic acid linkers, and the operation is simple and easy. Among them, the linking referred to in this disclosure can use a DNA ligase, the reaction conditions are mild, no adverse effects are imposed on subsequent reactions, and the operation is simple, and it is easy to carry out in batches.

When the synthetic strand is immobilized on the solid support by the 3′ end, the S3 includes: hybridizing the sequencing primers of the first region to be detected and the second region to be detected and a free 3′ end amplification primer on the first sequencing strand, and sequencing in segments or extending and linking into the whole synthetic strand; and immobilizing the 3′ end of the synthetic strand on the solid support by a cross-linking reaction. The cross-linking reaction can be a click chemical reaction, such as copper-catalyzed azide-alkyne cycloaddition reaction. In addition, in an implementation of this disclosure, a pre-denaturation procedure may be provided such that the first sequencing strand does not hybridize with P5 on the solid support.

Typically, the technical solutions of this disclosure can be applied to double-ended sequencing of nucleic acids. Usually, the single strand in a double-ended sequencing library sequentially includes a first linker sequence region linked to the solid support, a first index region, a first sequencing primer region, a sequence region to be detected, a second sequencing primer region, a second index region, and a second linker sequence region linked to the solid support from a 5′ end to a 3′ end. Correspondingly, the first region to be detected described in this disclosure may be a sequence to be detected with an unknown sequence, and the second region to be detected may be a first index region or a second index region. The index in sequencing is a tag sequence used to identify nucleic acid samples. During the sequencing process, nucleic acid samples are usually divided into small fragments, and each fragment needs to be labeled for subsequent data analysis.

In a typical implementation of this disclosure, hybridizing sequencing primers of the first region to be detected and the second region to be detected on the first sequencing strand, and sequencing in segments or extending and linking into a whole synthetic strand may include: phosphorylating a 5′ end of the sequencing primer of the first region to be detected, and phosphorylating both a 3′ end and a 5′ end of the sequencing primer of the second region to be detected; performing partial sequencing on the first region to be detected using a reversible terminator; extending the first sequencing strand as a template to form a segmented synthetic strand using a nucleic acid linker complementary to the 3′ end of the first sequencing strand on the solid support or the free 3′ end amplification primer (note: a polymerase cannot have 5′-3′ to exonuclease activity); linking the segments of the synthetic strand; and dephosphorylating the 3′ end of the sequencing primer of the second region to be detected, and sequencing or extending the second region to be detected to form the synthetic strand. Performing partial sequencing on the first region to be detected using a reversible terminator is because the synthetic strand for sequencing will generate residual scars after the cleavage of fluorescent groups, which will affect the sequencing of its complementary strands. Therefore, according to a typical implementation of this disclosure, the partial sequencing is adopted in this step instead of complete sequencing, leaving a portion of the template for natural synthesis, so that the complementary strands can be sequenced normally; and in a preferred embodiment of the present disclosure, a length range of the partial sequencing is 35-150 bp.

Since the sequencing synthetic strand in this disclosure is synthesized in segments and linked to form a whole synthetic strand, this method can be used in combination with other non-sequencing index detection methods. For example, the sequence of the index is preset, and the index is directly combined and distinguished by the probe carrying the label, that is, the sequence information of the index can be directly identified by the presence or absence of the label signal after the index is combined with the probe. In this way, the sequence information of the index can be identified only by the presence or absence of the fluorescence signal, which can greatly shorten the sequencing time and reduce the sequencing cost.

In an embodiment of this disclosure, there are multiple types of nucleic acids in the second region to be detected, and a probe that is reversely complementary to the multiple nucleic acids in the second region to be detected each carries a preset fluorescent label; and sequence information of the second region to be detected is identified by a fluorescence signal after hybridization between the probe and the second region to be detected. For example, the method of nucleic acid sequence detection for the second region to be detected includes: dividing the probe into N group(s), with the N≥1, where each group of the probe includes a type(s) of the probe, with the a≥1, and the a type(s) of the probe in the group each carries a different fluorescent label; hybridizing a first group of the probe carrying a fluorescent label with the nucleic acid of the second region to be detected to collect a fluorescent signal; stripping the first group of the probe; hybridizing a second group of the probe with the nucleic acid of the second region to be detected to collect a fluorescence signal; and repeating the above steps until all the second region to be detected is detected.

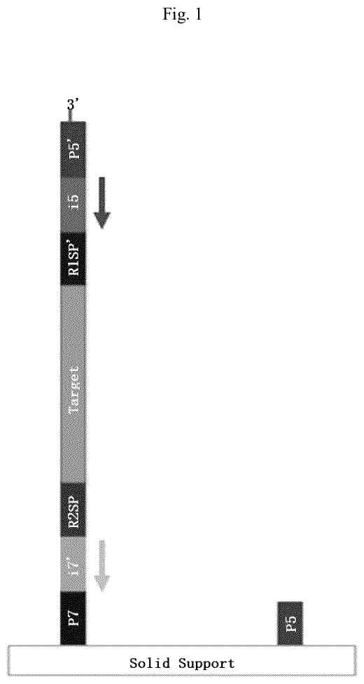

In an embodiment of the present disclosure, the construction of a DNA sequencing library includes: 1) randomly breaking the DNA to be detected into fragments of a certain length; and 2) processing the both ends of the fragments. As shown in FIG. 1, each resulting DNA single strand has P7, 17 index, and Rd2SP (Read2 Sequencing Primer) at the 5′ end, and P5′, i5 index, and Rd1SP (Read1 Sequencing Primer) at the 5′ end. These DNA single strands form a sample library.

The sequences of the above indexes are known, and the conventional index (1 or 2) is labeled with 4 fluorescent dyes (AF532, Rox, Cy5, and AF700). Probes that can bind to the indexes of known sequences are synthesized, and each probe carries a preset fluorescent label. When the fluorescent label after the index is bound to the probe emits a fluorescent signal, the sequence information of the index can be identified through the presence or absence of the fluorescent signal, i.e., which index it is can be identified.

Optionally, in other embodiments, the number of indexes (or samples) used is entirely identified by the implementer. For example, if the implementer wants to use 40 indexes, 10 rounds of hybridization (40 indexes divided by 4 fluorescence per round) are required to identify the sequence information of different indexes.

In the embodiment of FIG. 1, conventional i5 and i7 are used as index examples, 8 index probes can be used for i5 (1501, 1502, . . . , i508), requiring 2 rounds of hybridization (8 divided by 4), and 12 index probes can be used for i7 (1701, i702, . . . , i712), requiring 3 rounds of hybridization (12 divided by 4). The index probe is usually of 8 to 12 bp, and of course, it can be longer.

It can be understood that the use of AF532, Rox, Cy5, and AF700 for the fluorescent label described above is only illustrative, and other dyes or fluorescent labels can also achieve this purpose.

In the implementation of the sequencing method of this disclosure, other steps can be achieved using some conventional technical means in the art. However, in order to improve the overall efficiency and accuracy of sequencing, preferably, removing an original template strand or removing a complementary strand of the original template strand includes: cleaving a uracil base on a linker sequence on the solid support to form a nick, and removing the original template strand and the complementary strand of the original template with the nick using a formamide solution; and typically, the uracil base can be cleaved using a uracil hydrolase, or the 8-oxoguanine base can be cleaved using a Fpg glycosidase, and a phosphate group is generated at a 3′ end. The probe can be stripped with a formamide solution, and then whether the probe is completely stripped is identified by fluorescence observation.

The beneficial effects of this disclosure will be explained in further detail below in conjunction with specific embodiments. Operations or reagents not described in detail in this disclosure may be achieved using conventional technical means or reagents in the art.

### Embodiment 1

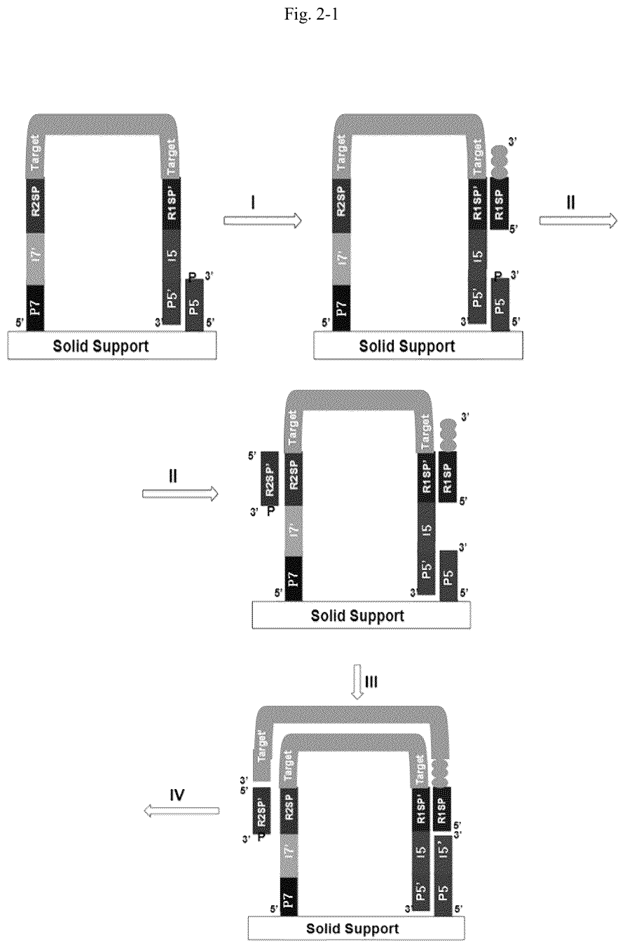

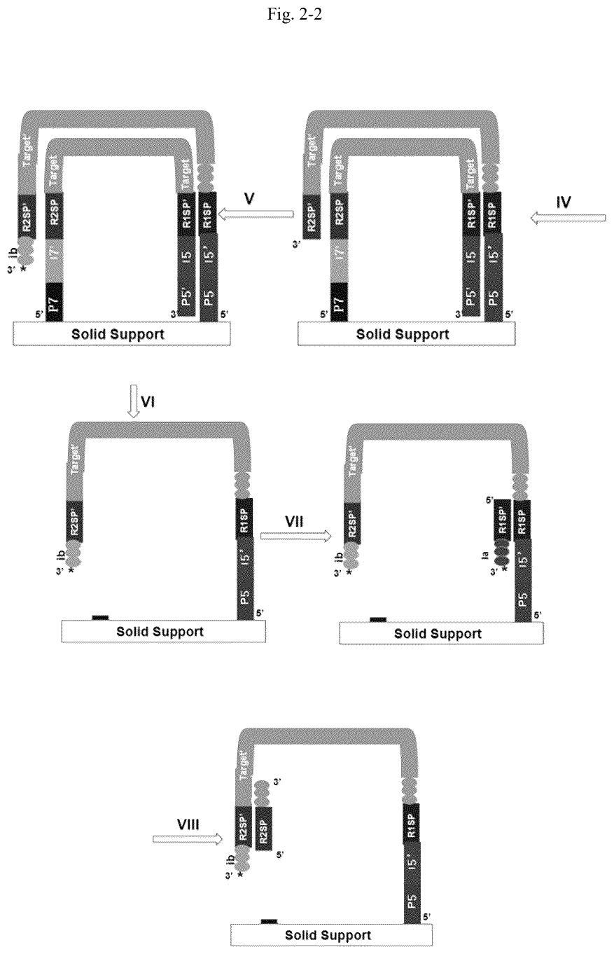

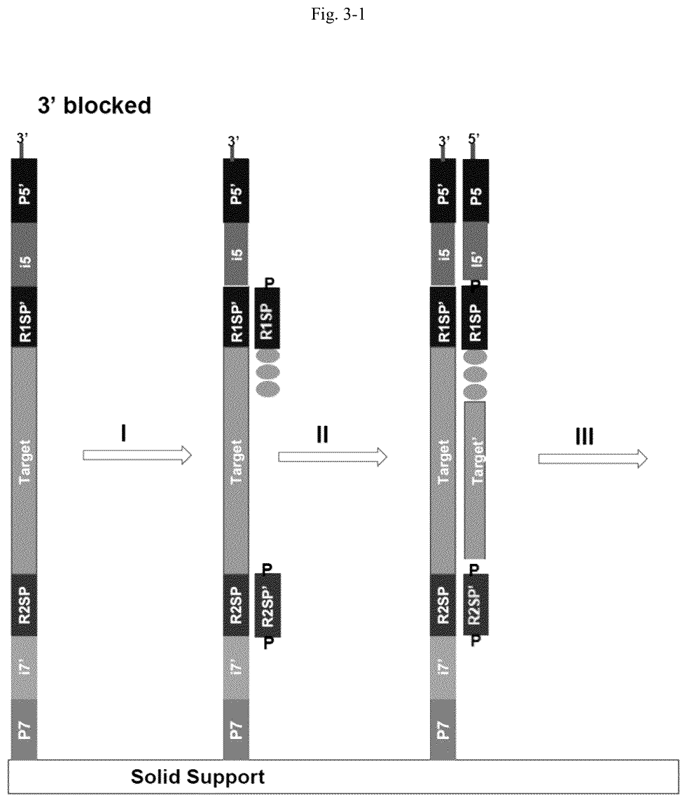

For a method of sequencing in this embodiment, please refer to FIGS. 2-1 and 2-2 for steps.
Step 1, forming a cluster through typical bridge amplification. A uracil base was cleaved on P5, and a nick was formed on the P5 strand. Conditions: 40 U/mL enzyme, 3° C. (6 minutes twice) and 41° C. (6 minutes twice) (note: total incubation time was 24 minutes). The strand with a nick was removed from a flow cell with formamide. Note: in this embodiment, a uracil hydrolase was used, which produced a fragment with a phosphate group at a 3′ end.

After the step 1, the remaining P5 on a solid support was automatically coupled to the uncleaved strand (e.g., under room temperature buffer conditions), without the need for additional hybridization steps.
Step 2, hybridizing R1SP (phosphorylated at the 5′ end) with the cluster, and sequencing with a reversible terminator (a length range of sequencing was 35-150 bp).
Step 3, removing the phosphate group at the 3′ end of P5 using a phosphatase rSAP (NEB). Conditions: 16 U/mL rSAP, 55° C., 30 minutes. R2SP′ (phosphorylated at both 3′ and 5′ ends) was hybridized with the cluster.
Step 4, extending to form a segmented synthetic strand with P5 and RISP as primers (note: polymerase could not have 5′-3′ exonuclease activity).
Step 5, repairing the nick, and linking segments in the synthetic strand to form a whole synthetic strand.
Step 6, dephosphorylating the 3′ end of R2SP′, performing index 7 sequencing with R2SP′ as a primer, and then blocking the 3′ end;
Step 7, hydrolyzing an oxG on a P7 sequence with a Fpg glycosidase, resulting in a nick on the strand. Conditions: 230 U/mL Fpg (NEB), 3° C., 30 minutes. The cluster was peeled off with the formamide solution so that it only had complementary strands.
Step 8, hybridizing RISP′ with the cluster, sequencing index 5 using the reversible terminator, and then stripping RISP′ and index 5.
Step 9, hybridizing R2SP with the cluster, and sequencing using the reversible terminator.

### Embodiment 2

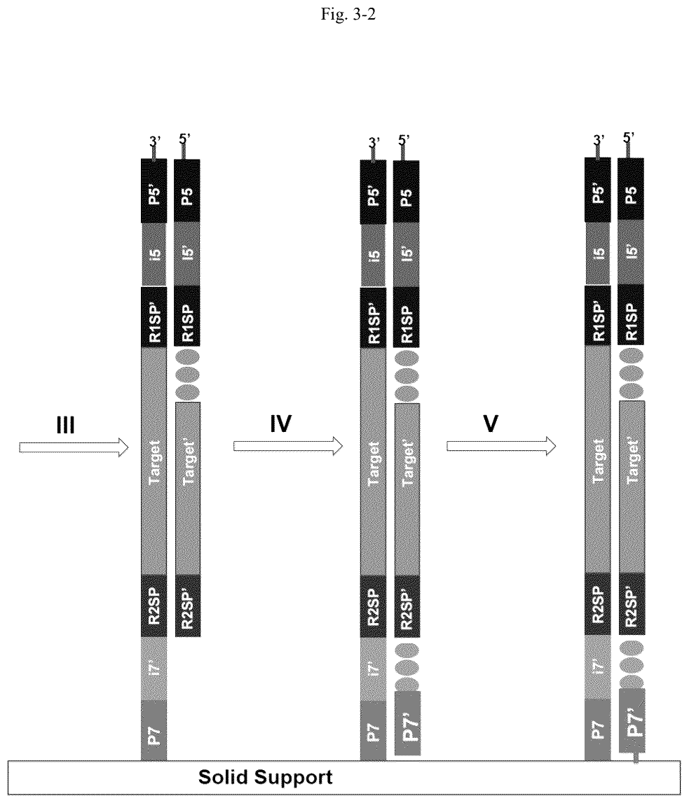

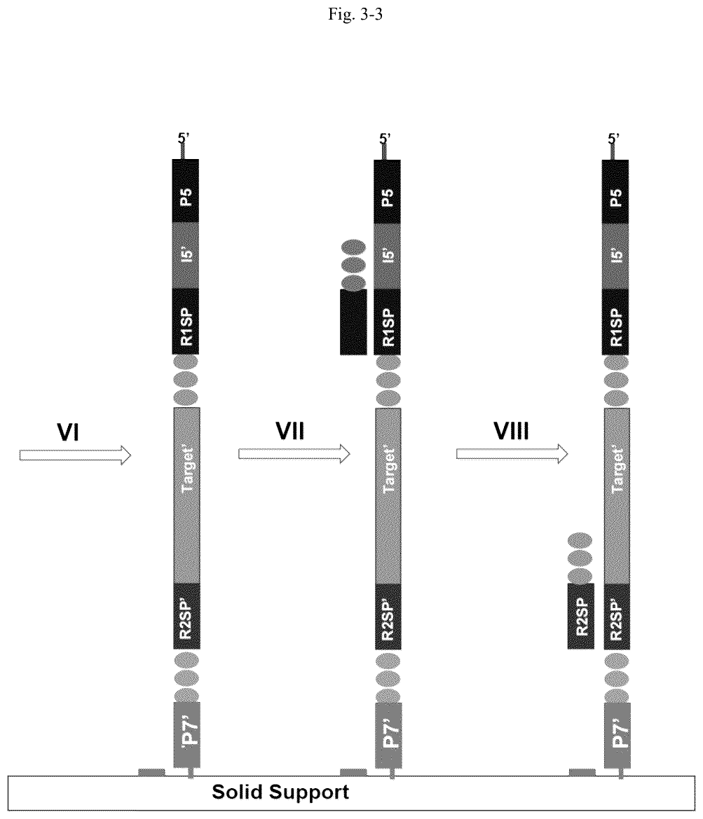

For a method of sequencing in this embodiment, please refer to FIGS. 3-1, 3-2, and 3-3 for steps.
Step 1, forming a cluster through typical bridge amplification. A uracil base was cleaved on P5 (in this embodiment, the uracil base modified on P5 was located 1-5 bp near a 5′ end), and a nick was formed on the P5 strand. Conditions: 40 U/mL enzyme, 3° C. (6 minutes twice) and 41° C. (6 minutes twice) (note: total incubation time was 24 minutes). The strand with a nick was removed from a flow cell with formamide. Note: in this embodiment, a uracil hydrolase was used, which produced a fragment with a phosphate group at a 3′ end.
Step 2, hybridizing R1SP (phosphorylated at the 5′ end) and R2SP′ (phosphorylated at both 3′ and 5′ ends) with the cluster, and sequencing with a reversible terminator (a length range of sequencing was 35-150 bp).
Step 3, hybridizing P5, and extending P5 and RISP using a polymerase without 5′-3′ exonuclease activity.
Step 4, repairing the nick, and linking segments in a synthetic strand.
Step 5, dephosphorylating the 3′ end of R2SP′, extending R2SP to sequence index 7, and continuing to extend to form P7′.
Step 6, introducing functional groups at a 3′ end of the synthetic strand, and linking them to a solid support. For example, an azide (N3) group could be introduced into the 3′ end of the synthetic strand with a terminal transferase, which can react with alkyne on the solid support (hydrogel), so that the 3′ end was linked to the solid.
Step 7, hydrolyzing an oxG on a P7 sequence with Fpg (in this embodiment, the oxG base modified on P7 was located 1-5 bp near a 5′ end), resulting in a nick on the strand. Conditions: 230 U/mL Fpg (NEB), 3° C., 30 minutes. The cluster was peeled off with the formamide solution so that it only had complementary strands.
Step 8: hybridizing RISP′, sequencing index 5 using the reversible terminator, and then stripping RISP′ and index 5.
Step 9, hybridizing R2SP, and sequencing using the reversible terminator.

### Embodiment 3

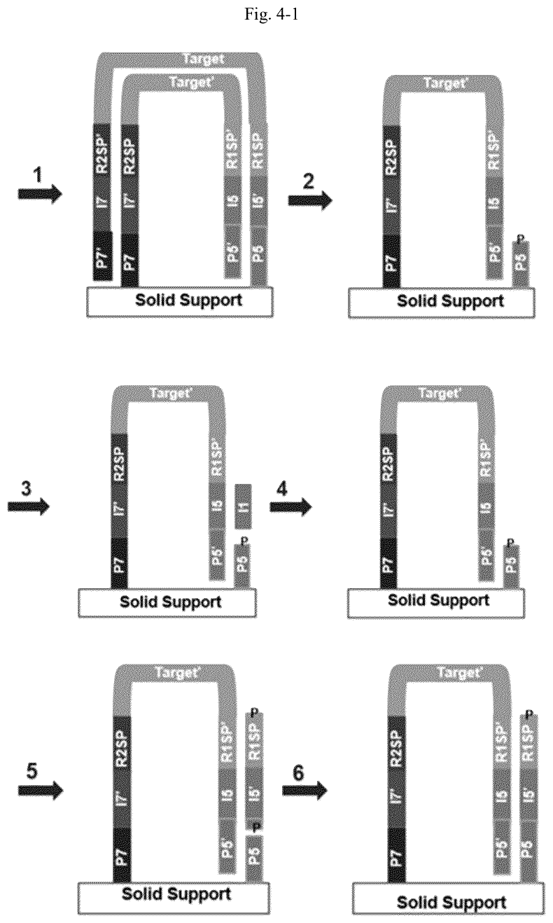

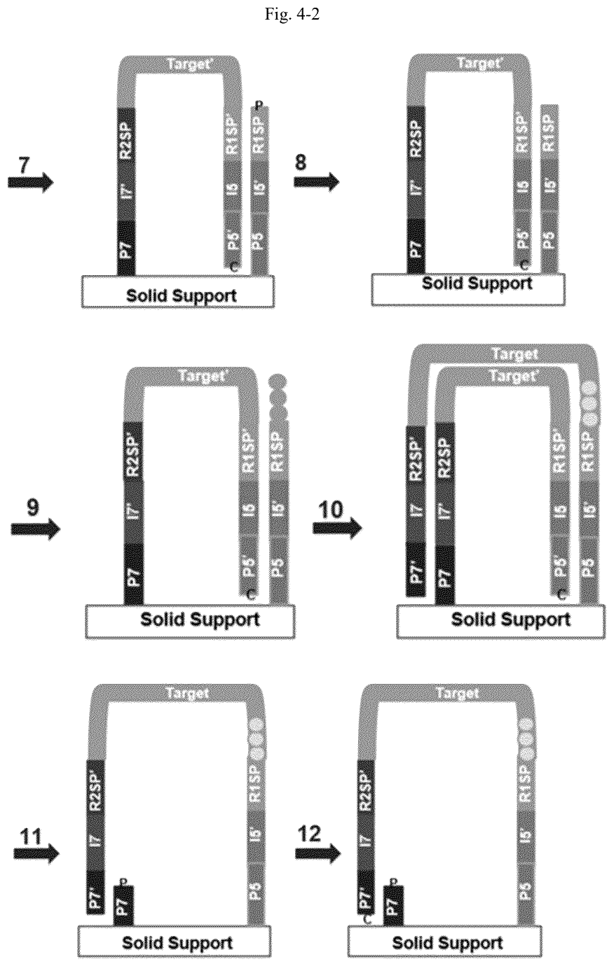

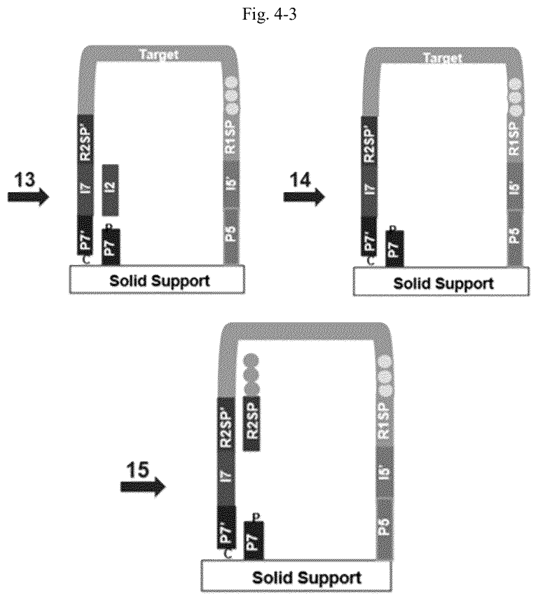

For a method of sequencing in this embodiment, please refer to FIGS. 4-1, 4-2 and 4-3 for steps.
Step 1, forming a cluster through typical bridge amplification.
Step 2, cleaving a uracil base on P5 and form a nick on the P5 strand. Conditions: 40 U/mL enzyme, 3° C. (6 minutes twice) and 41° C. (6 minutes twice) (note: total incubation time was 24 minutes). The strand with a nick was removed from a flow cell with formamide. Note: in this embodiment, a uracil hydrolase was used, which produced a fragment with a phosphate group at a 3′ end (marked as P in FIGS. 4-1, 4-2 and 4-3).
Step 3, identifying index 5 sequencing with an index probe (11).
Step 4, stripping the index probe with the formamide solution. During the imaging process, no fluorescent cluster was observed, confirming the completion of stripping.
Step 5, removing the phosphate group at the 3′ end using a phosphatase rSAP (NEB). Conditions: 16 U/mL rSAP, 55° C., 30 minutes. The oligonucleotide covering a small portion of P5, index 7, and RISP was hybridized with the cluster, and both a 3′ end and a 5′ end of the oligonucleotides were phosphorylated.
Step 6, linking the oligonucleotide to the 3′ end of P5 on a solid support. Conditions: a 20 U/μL Hi-T4 DNA ligase (NEB) reacted with 2 mM ATP at 25° C. for 1 hour.
Step 7, blocking all free 3′-hydroxyl groups with a terminator or phosphorylation, where the terminator did not have any free 3′-hydroxyl groups, and for example, could be a deoxyribonucleotide.
Step 8, removing the phosphate groups on RISP under the same conditions as in the step 5.
Step 9, sequencing with a reversible terminator (a length range of sequencing was 35-150 bp). In addition, the 5′ end of the sequencing strand was covalently bound to the solid support, as confirmed by stripping with the formamide solution by the inventor (i.e., unable to remove cluster fluorescence). Because if the sequencing strand was not covalently bound, the fluorescence of the cluster was eliminated during sequencing when incubated at 55-70° C.
Step 10, fully extending the sequencing strand with an enzyme after removing the protective group at the 3′ end. Conditions: 320 U/mL enzyme and 120 μM dNTP, temperature 65° C., 1 hour.
Step 11, hydrolyzing an oxG on the P7 sequence with Fpg, resulting in a nick on the strand. Conditions: 230 U/mL Fpg (NEB), 3° C., 30 minutes. The cluster was peeled off with the formamide solution so that it only had complementary strands.
Step 12, terminating all free 3′-hydroxyl groups under the same conditions as in the step 7.
Step 13, identifying index 7 by the index probe (12) using the same method as in the step 3.
Step 14, stripping the index probe with the formamide solution. When the index probe was washed off (such as with formamide), the P7 and P′7 parts would dissociate, but when the formamide was washed off with a buffer, the temperature was lower than 70° C., and P7 and P′7 hybridized automatically.
Step 15, sequencing Read 2 using reversible terminator sequencing (a sequencing length range was 35-150 bp).

### Embodiment 4

Sequencing was performed using the method of Embodiment 1:
1) hybridizing a sequencing primer to a cluster;
2) incorporating a reversible terminator (four nucleotide bases A, T, G and C modifying four fluorescent dyes, AF532, Rox, Cy5 and AF700, respectively) into the sequencing strand/primer;
3) taking images of 4 channels (AF532 channel, Rox channel, Cy5 channel, and AF700 channel), and decoding for incorporated bases of each cluster;
4) cleaving a protective group on 3′-OH together with a fluorescent dye from the incorporated reversible terminator;
5) completing a sequencing cycle with steps 2), 3), and 4), and continuing the cycle and decoding the sequence for each cluster.

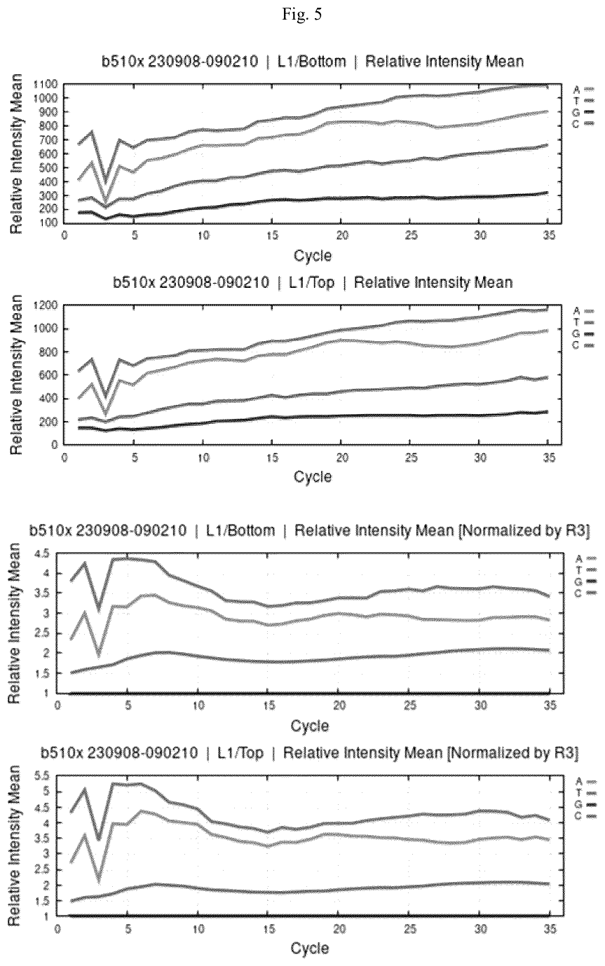

FIG. 5 shows relationships between a cluster average fluorescence intensity and an SBS cycle for four channels (A, G1 channel; T, G2 channel; G, R3 channel; C, R4 channel). From top to bottom, in the first image, read Read 1 from the bottom of the flow cell; in the second image, read Read 1 from the top of the flow cell, and in the third image, read Read 2 from the bottom of the flow cell; and in the forth image, read Read 2 from the top of the flow cell.

The specific results are shown in Table 1.

TABLE 1

| | Read 1 | Read 2 |
|---|---|---|
| Read Length (bp) | 35 | 35 |
| Yield | 7,725,344 | 10,428,704 |
| Error Rate (%) | 0.19 | 0.20 |
| Q30 (%) | 80.15 | 79.62 |

The foregoing is only a preferred embodiment of the present disclosure and is not intended to limit the present disclosure. For those skilled in the art, the present disclosure may have various modifications and variations. Any modifications, equivalent substitutions, improvements, etc. made within the spirit and principles of the present disclosure shall be included within the scope of protection of the present disclosure.

1. A method of nucleic acid sequencing, comprising:
S1, providing a solid support with at least two types of nucleic acid linkers and a single-stranded template nucleotide, wherein both ends of the single-stranded template nucleotide have sequences that are complementary to the nucleic acid linkers and the single-stranded template nucleotide has a region to be detected, respectively, and the region to be detected comprises a first region to be detected and a second region to be detected;
S2, forming multiple nucleotide strands identical or complementary to the single-stranded template nucleotide on the solid support by a bridge amplification reaction, removing one of the nucleotide strands identical or complementary to the single-stranded template nucleotide by cleaving, and retaining a first sequencing strand; and
S3, hybridizing sequencing primers of the first region to be detected and the second region to be detected on the first sequencing strand, and sequencing in segments or extending and linking into a whole synthetic strand.

2. The method according to claim 1, wherein the synthetic strand is immobilized to the solid support by a 5′ end or a 3′ end.

3. The method according to claim 2, wherein when the synthetic strand is immobilized on the solid support by the 5′ end, the S3 comprises:
hybridizing a 3′ end of the first sequencing strand to the nucleic acid linkers on the solid support;
hybridizing the sequencing primers of the first region to be detected and the second region to be detected on the first sequencing strand, and sequencing in segments or extending and linking into the whole synthetic strand, wherein the linking comprises linking the nucleic acid linkers on the solid support to other segments of the synthetic strand.

4. The method according to claim 2, wherein when the synthetic strand is immobilized on the solid support by the 3′ end, the S3 comprises:
hybridizing the sequencing primers of the first region to be detected and the second region to be detected and a free 3′ end amplification primer on the first sequencing strand, and sequencing in segments or extending and linking into the whole synthetic strand; and
immobilizing the 3′ end of the synthetic strand on the solid support by a cross-linking reaction.

5. The method according to claim 1, wherein the method of nucleic acid sequencing is a double-ended sequencing method, the first region to be detected is a sequence to be detected with an unknown sequence, and the second region to be detected is a first index region or a second index region.

6. The method according to claim 5, wherein in the S3, the step of hybridizing sequencing primers of the first region to be detected and the second region to be detected on the first sequencing strand, and sequencing in segments or extending and linking into a whole synthetic strand comprises:
phosphorylating a 5′ end of the sequencing primer of the first region to be detected, and phosphorylating both a 3′ end and a 5′ end of the sequencing primer of the second region to be detected;
performing partial sequencing on the first region to be detected using a reversible terminator, preferably with a length of 35-150 bp for the partial sequencing;
extending the first sequencing strand as a template to form a segmented synthetic strand using a nucleic acid linker complementary to the 3′ end of the first sequencing strand on the solid support or the free 3′ end amplification primer;
linking the segments of the synthetic strand; and
dephosphorylating the 3′ end of the sequencing primer of the second region to be detected, and sequencing or extending the second region to be detected to form the synthetic strand.

7. The method according to claim 1, wherein the single-stranded template nucleotide sequentially comprises a first linker sequence region linked to the solid support, a first index region, a first sequencing primer region, a sequence region to be detected, a second sequencing primer region, a second index region, and a second linker sequence region linked to the solid support from a 5′ end to a 3′ end; and the method comprises:
immobilizing the single-stranded template nucleotide on the solid support through a bridge reaction to form a cluster, removing one of nucleotide strands identical or complementary to the single-stranded template nucleotide by cleaving, and retaining a first sequencing strand;
hybridizing a probe carrying a fluorescent label for identifying the first index region with a nucleic acid of the first index region to collect a fluorescent signal and identify a sequence of the first index region, and then stripping the probe;
hybridizing a first nucleic acid fragment covering a portion of the first linker sequence region, the first index region and the first sequencing primer region with the cluster, linking the first nucleic acid fragment to a linker sequence on the solid support, and partially sequencing the sequence to be detected with the first nucleic acid fragment as a primer, and then extending to the second linker sequence region;
removing the first sequencing strand;
hybridizing a probe carrying a fluorescent label for identifying the second index region with a nucleic acid of the second index region to collect a fluorescent signal and identify a sequence of the second index region, and then stripping the probe; and
performing complementary strand sequencing of the sequence to be detected using a second nucleic acid fragment complementary to the second sequencing primer region as a primer.

8. The method according to claim 1, wherein the removing one of nucleotide strands identical or complementary to the single-stranded template nucleotide by cleaving comprises: cutting a uracil base or an 8-oxoguanine base on a nucleic acid linker on the solid support to form a nick, and removing a nucleotide strand with the nick using a formamide solution.

9. The method according to claim 8, wherein the uracil base is cleaved using a uracil hydrolase, or the 8-oxoguanine base is cleaved using an Fpg glycosidase.

10. The method according to claim 7, wherein both a 5′ end and a 3′ end of the first nucleic acid fragment are phosphorylated.

11. The method according to claim 10, wherein the step of linking the first nucleic acid fragment to a linker sequence on the solid support, and sequencing the sequence to be detected with the first nucleic acid fragment as a primer specifically comprises:
linking the first nucleic acid fragment to the linker sequence on the solid support using a DNA ligase, removing phosphorylation of the 3′ end of the first nucleic acid fragment using a phosphatase, and sequencing the sequence to be detected with the first nucleic acid fragment as a primer.

12. The method according to claim 7, wherein after the removing the first sequencing strand, the method further comprises a step of blocking a free 3′-hydroxyl group with a terminator or phosphorylation, and optionally, the terminator is a dideoxynucleotide.

13. The method according to claim 7, wherein sequences of the probe for identifying the first index region and the probe for identifying the second index region are known, and the probe each carries a preset fluorescent label.

14. The method according to claim 7, wherein the step of hybridizing a probe carrying a fluorescent label for identifying the first index region with a nucleic acid of the first index region to collect a fluorescent signal and identify a sequence of the first index region, or the step of hybridizing a probe carrying a fluorescent label for identifying the second index region with a nucleic acid of the second index region to collect a fluorescent signal and identify a sequence of the second index region comprises:
S1, dividing the probe into N group(s), with the N≥1, wherein each group of the probe comprises a type(s) of the probe, with the a≥1, and the a type(s) of the probe in the group each carries a different fluorescent label; and hybridizing a first group of the probe carrying a fluorescent label with the nucleic acid of the first index region to collect a fluorescent signal;
S2, stripping the first group of the probe, and hybridizing a second group of the probe with the nucleic acids of the first index region or the second index region to collect a fluorescence signal; and
S3, repeating the step S2 until all the first index region or the second index region is detected.

15. The method according to claim 7, wherein stripping the probe comprises stripping the probe with a formamide solution; and
preferably, it further comprises determining whether the probe is completely stripped by fluorescence observation.

16. A nucleic acid sequencing kit, comprising a relevant reagent for nucleic acid sequencing and a DNA ligase; and
preferably, the relevant reagent for nucleic acid sequencing comprises a sequencing primer, a polymerase, and dNTP.

17. A nucleic acid sequencing apparatus, comprising a control unit and a sequencing unit, wherein the control unit controls the sequencing unit to perform the method of nucleic acid sequencing according to claim 1.
